(19) United States
(12) Patent Application Publication
(10) Pub. No.: US 2006/0252938 A1
(43) Pub. Date: Nov. 9, 2006

(54) Process for the separation of palladium catalyst from crude reaction mixtures of aryl acetic acids obtained by carbonylation

(75) Inventors: Xavier Sava, Mannheim (DE); Michael Röper, Wachenheim (DE); Colin Orgill, Corpus Christi, TX (US); John Cooper, Corpus Christi, TX (US)

Correspondence Address: CONNOLLY BOVE LODGE & HUTZ LLP, P.O. BOX 2207, WILMINGTON, DE, 19899-2207, US

(73) Assignee: BASF AKTIENGESELLSCHAFT, Ludwigshafen (DE)

(21) Appl. No.: 10/554,247

(22) PCT Filed: Apr. 16, 2004
(86) PCT No.: PCT/EP04/04047

§ 371 Date: Oct. 25, 2005

Related U.S. Application Data
(60) Provisional application No. 60/466,123, filed on Apr. 28, 2003.

(51) Int. Cl. B01J 38/00; C07C 51/00; C07D 263/52; C07D 209/82

(52) U.S. Cl. 548/217; 502/20; 548/444; 562/406

(57) ABSTRACT

A process for the separation of palladium from solvent-free at room temperature solid crude reaction mixtures comprising aryl acetic acids of the general formula (I), wherein Z means phenyl, napht-2-yl, 9H-fluoren-2-yl, substituted carbazol-2-yl, benzoxazol-5-yl, either of which can be substituted with H, C₁-C₈-alkyl optionally cyclic and optionally substituted with —F or —Cl, C₆-C₁₀-aryl optionally substituted with F or Cl OR⁴, COR⁵, F, —Cl; optionally substituted pyrrolyl or dehydropyrrolyl or 1-oxo-1,3-dehydro-isoindol 2-yl; R¹ means H or C₁-C₄-alkyl; R², R³, R⁴, R⁵ mean independently of each other H, C₁-C₈ alkyl, C₆-C₁₀-aryl optionally substituted with —F or Cl, or thiophenyl; which crude mixture is obtained by palladium catalyzed carbonylation, by adsorption of the palladium on solid adsorbents, characterized in that the adsorption is carried out in the absence of a reducing agent for palladium and at a temperature, where the crude reaction mixture is molten.

The present invention relates to a process for the separation of palladium from solvent-free, at room temperature solid crude reaction mixtures of aryl acetic acids of the general formula

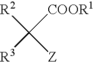

wherein

Z means phenyl, napht-2-yl, 9H-fluoren-2-yl, carbazol-2-yl, benzoxazol-5-yl, either of which can be substituted with H, C₁-C₈-alkyl optionally cyclic and optionally substituted with —F or —Cl, C₆-C₁₀-aryl optionally substituted with —F or —Cl, —OR⁴, —COR⁵, —F, —Cl, optionally substituted pyrrolyl or dehydropyrrolyl or 1-oxo-1,3-dehydro-isoindol-2-yl R¹ means H or C1-C4-alkyl, R², R³, R⁴, R⁵ mean independently of each other H, C₁-C₈ alkyl, C₈-C₁₀-aryl optionally substituted with F or Cl, thiophenyl,

which is obtained by palladium catalyzed carbonylation, by absorption of the palladium on solid adsorbents.

Many processes for the palladium catalyzed carbonylation are known in the chemical industry.

Of particular interest is the carbonylation of alkyl aromatic substrates such as (substituted) styrenes, vinylnaphthalenes, benzyl alcohols, benzyl halides, 1-naphthyl-alcohols, 1-naphthyl-alkyl-halides and the like. The carboxylic acid derivatives find use as fine chemicals for instance in the pharmaceutical industry.

However the recovery of palladium from such processes is in many cases unsatisfactory and adds to (strains) the production costs. In addition, residual heavy metal traces in the products are not acceptable for many applications, and need to be removed by costly purification processes.

Ibuprofen for example can be obtained with a good selectivity through the carbonylation of 1-(4′-isobutylphenyl)-ethanol in an acidic medium. The carbonylation can proceed in the presence of (1) a catalyst consisting essentially of a palladium compound complexed with at least one acid stable, monodentate phosphine ligand; (2) dissociated hydrogen ions from an acid which substantially completely ionizable in a dilute aqueous solution, and; (3) dissociated halide ions (EP 400 892). After the reaction palladium is recovered as palladium black at many places of the purification-process, which is described in the patent EP-474,509. This makes precisely the complete recovery of palladium difficult and unpractical.

In EP 337 803 a process has been described to recover the palladium catalyst from a crude ibuprofen solution: a non-coordinating solvent is added to the organic phase, provoking the precipitation of the palladium catalysts, optionally with concomitantly removing any solvents from the organic phase. This process presents the disadvantage of involving an organic solvent, which has to be then removed. Moreover, by low palladium loading complete precipitation becomes more and more difficult.

In the literature, several patents describe the recovery of Group VIII noble metals by adsorption on solid supports, like activated carbon or ion exchange resins.
GB-2127001 describes the recovery of precious metal (e.g. Au, Ag, Pt, Pd) through for example, adsorption on activated carbon fiber. The concerned extracted mixture are here aqueous solutions.
JP 60-231630 and JP 60-237046 describe the recovery of palladium metal from an oxidative carbonylation reaction. The neutral cinnamic ester-containing mixture is treated with a carbon carrier (preferably activated carbon) to separate and recover the metallic palladium effectively.
EP 552 846 discloses the recovering of precious metal from non-aqueous effluent, wherein the effluent is contacted with a reduction agent being a lower olefin or carbon monoxide, the precious metal is deposited onto a carrier comprising carbonaceous combustible material, and the precious metal loaded carrier is separated from the effluent. When coordinative ligands like triphenylphosphine are present in the effluent, a substantial inactivation of this ligand through oxidation, prior to contacting with the reducing agent, is proposed.
In this patent is to point out the necessity of using olefin or carbon monoxide as reduction agent to make palladium adsorption onto the carrier easier.
The use of hydrogen peroxide to inactivate triphenylphosphine may conduct in some cases to a concurrent oxidation of the product.
EP 049 807 discloses the recovery of a salt of Group VIII noble metal from dilute acidic solution of pH less than 4, comprising the steps of (i) adding to the dilute solution up to 10% by weight hydrogen peroxide, (ii) contacting the resulting per-oxide-containing solution with activated carbon and adsorbing on the activated carbon the salt of the Group VIII metal, and (iii) separating this activated carbon.
Under these conditions this process is rather adapted to the recovery of Group VIII noble metal from water containing homogeneous solutions. Moreover the use of excess of hydrogen peroxide may conduct to the formation of side products in the case of benzylic acids.
WO 02/051783 discloses the hydroxycarbonylation of an organic compound comprising a conjugated unsaturated bond, through the action of carbon monoxide in the presence of a palladium catalyst and of a compound which is not soluble in the reaction medium, such as activated carbon, silica gel, alumina, etc . . . . The resulting reaction medium is treated with hydrogen to reduce palladium to zero oxidation state and to help by the deposition of palladium on the insoluble support. Palladium is then recovered by filtering off the solid support. The treatment with hydrogen to reduce palladium to the oxidation state zero, easing its adsorption on activated carbon for example, implies to operate under pressure (typically 20 bar), which generates higher complexity and costs.
GB 1 321 275 describes a method for recovering rhodium carbonyl catalyst from a crude oxo reaction mixture, by adsorption on a basic ion exchange resin.
U.S. Pat. No. 4,388,279 discloses the recovery of metal catalyst traces, such as rhodium, from reaction mixtures by adsorption on a solid adsorbent such ion-exchange resins, molecular sieves, or a metal compound of Groups IA or IIA of the Periodic Table.
WO 02/33135 claims a method for the recovery of a metal from a liquid medium, through the adsorption on a functionalized polymer fiber capable of binding the metal. The adsorption is carded out in an organic solvent or In an aqueous mixture of an organic solvent.

It is an object of the present invention to develop an improved method to separate palladium from the crude reaction mixtures defined above.

We have found that this object is achieved by a process for the separation of palladium from solvent-free, at room temperature solid crude reaction mixtures comprising aryl acetic acids as defined above and obtained by palladium catalyzed carbonylation by adsorption of the palladium on solid adsorbents, which is characterized in that the adsorption is carried out in the absence of a reducing agent for palladium and at a temperature, where the crude mixture is molten.

The present invention relates to a method of separating palladium directly from crude reaction mixtures substituted of aryl acetic acid products or esters thereof, which are obtained by Pd-catalyzed carbonylation of olefins (styrenes, vinylnaphthalenes and the like), benzyl alcohols, benzyl halides, 1-naphthyl-alcohols or 1-naphthyl-alkyl-halides.

Such products are preferably selected from the group consisting of ibuprofen, naproxen, ketoprofen, flurbiprofen, indoprofen, suprofen, hexaprofen, pirprofen, fenoprofen, cicloprofen, mexoprofen, benoxaprofen, and carprofen.

Methods for obtaining such crude product mixtures are known in the art, e.g. from EP-A 400 892. The mixtures are obtained in such a way that the organic phase consisting of the desired aryl acetic acid product and by-products is separated from the aqueous reaction medium and, if desired, may be subsequently dried over sodium sulfate.

The crude reaction mixture is acidic with a pH-value in the range of from 0 to 5 and solid at ambient temperature (20° C.). The acidity of the mixture stabilizes palladium in the oxidation state two.

Separation of palladium from the crude product is achieved by adsorption of the palladium on different solid adsorbents, selected from the group consisting of activated carbon, optionally functionalized silica gel or alumina, infusorial earth, magnesium oxide, ion-exchange resin, neutral solid adsorbent, zeolite, or a combination of two or more of such adsorbents.

The adsorption is carried out in the absence of an reducing agent for palladium such as hydrogen, carbon monoxide, formic acid or an olefine.

Inactivation of triphenylphosphine ligand by contacting the mixture with oxygen of air, or by addition of oxygenated water can be avoided.

According to one preferred embodiment palladium removal is achieved by adsorption on activated carbon. More preferably an activated carbon with an average particle size <150 μm for 80% of the particles is chosen. Even more preferred is an average particle size of <60 μm for 80% of the particles.

In another preferred embodiment palladium removal is achieved by adsorption on basic ion-exchange resins, more preferably on strong basic ion exchange resins carrying functional groups such as quaternary ammonium groups. Such resins are for instance composed of Styrene-Divinylbenzene copolymers functionalized with quaternary ammonium groups in the form of the chlorides and are commercially available under the trade name Amberlite® (Rohm and Haas Co.) or Amberjet©. Especially suited are Amberlite IRA-Grade “Gel”-Type resins.

According to another preferred embodiment functionalized silica gels can be used. Such gels can be functionalized with phosphine or for example diphenylphosphine groups.

The adsorption is carried out at a temperature where the reaction mixture is molten, i.e. in the range of 40° C. to 200° C., preferably from 40° C. to 150° C., more preferably 60 to 120° C.

Typical amounts of adsorbents used are 0.05 to 10%, preferably 0.1 to 5%, relative to the weight of the reaction mixture.

The adsorbents can be added directly to the crude reaction mixture. The mixture is then stirred at the chosen temperature.

The time of treatment is depends on the type of adsorbent used. When activated carbon is used as an adsorbent appropriate times of treatment can range from several minutes to several hours depending on the batch size.

If ion exchange resins are used, it may be preferred to pass the crude reaction mixture over a bed of such resin with a flow rate of 0.5 to 10 m/hour, preferably 1 to 5 m/hour.

The adsorption can be carried out at normal atmospheric pressure, when the mixture is padded through a bed of an adsorbent it is also possible to use slightly elevated pressures.

Preferably the adsorption is carried out under a protective atmosphere. Suitable protective gases are for Instance argon or nitrogen. Carbon monoxide may also be used as protective gas.

The adsorbent can be easily separated from the crude reaction mixture by filtration. The palladium can then be recovered from the adsorbent by conventional methods.

The method according to the present invention offers several advantages over the methods known from the prior art. As mentioned above the use of reducing agents for palladium or the Inactivation of the triphenylphosphine ligands can be avoided. In addition to that the method can be carried out at normal pressure and in the absence of solvents. Palladium levels are effectively lowered in an easy and economic manner.

### EXAMPLES

Crude ibuprofen used in the following examples was obtained by a carbonylation reaction as described in EP-A-400,892. Reaction conditions were chosen among those, which conduct to a reaction mixture, containing more than 90% by weight ibuprofen. Palladium concentration in crude ibuprofen was measured by means of ICP-MS.

A typical ibuprofen mixture was obtained as follows:

1-(4′-Isobutylphenyl)ethanol (IBPE) (50.0 g, 280 mmol), PdCl₂ (3.3 mg, 0.019 mmol), PPh₃ (60 mg, 0.23 mmol) and 26% HCl (25 g) were charged to a 300 ml Hastelloy C autoclave which was sealed and purged with N₂ and CO. The autoclave was pressured to 165.5·10⁵ Pa with CO and the contents were heated to 130° C. for 3 h with stirring. The autoclave was cooled to room temperature, vented of CO, and the sample was collected. The organic layer was separated from the aqueous layer. The products were analyzed by GLC. Conversion (IBPE)=99% and selectivity (ibuprofen)=95.8%. Palladium concentration in crude ibuprofen: 33 ppm.

### Example 1

A 30 g sample of a crude Ibuprofen mixture obtained as previously described was heated in a Schlenk tube at a temperature of about 70° C. 0.6 g of activated carbon (with an average particle-size <40 μm for 80% of the particles) was added to the mixture. The mixture was then stirred for 1 h at 70° C., and filtered through a glass filter N 4. Palladium concentration in crude ibuprofen as well as the operating conditions are summarized in table 1.

### Example 2-4

The same procedure as in example 1 is followed, except that the amount of activated carbon used, as well as the operating conditions may be different. These data are presented in the table 1, together with the results of the palladium-concentration analysis.

TABLE 1
Adsorption of palladium on activated carbon

| | | Quantity^{a)} | Temperature | Time | Pd residual^{b)} |
|---|---|---|---|---|---|
| Example | Adsorbent | (%) | (° C.) | (h) | (%) |
| 1 | Activated | 2 | 70 | 1 | 7 |
| | carbon | | | | |
| 2 | Activated | 3 | 90 | 1 | 4 |
| | carbon | | | | |
| 3 | Activated | 2 | 110 | 1 | 6 |
| | carbon | | | | |
| 4 | Activated | 1 | 90 | 1 | 15 |
| | carbon | | | | |

^{a)}Amount of adsorbent relative to the mixture, in weight percent.
^{b)}Amount of palladium remaining in ibuprofen mixture in comparison to initial Pd amount.

### Comparative Example A

The same procedure as in examples 1-4 has been followed, but with no addition of activated carbon. Results concerning the palladium concentration and the operating conditions are presented in table 2.

TABLE 2
Comparative example

| | | Quantity | Temperature | Time | Pd residual |
|---|---|---|---|---|---|
| Example | Adsorbent | (%) | (° C.) | (h) | (%) |
| A | — | — | 90 | 1 | 90 |

### Example 5-9

Analog procedures as followed in Ex 1-4 have been followed, except that activated carbon was replaced by a strong basic ion-exchange resin or by a silica gel functionalized with diphenylphosphine groups. Results concerning the palladium concentrations and the detailed operating conditions are presented in table 3.

TABLE 3
Adsorption of palladium on basic ion-exchange resins

| | | Quantity | Temperature | Time | Pd residual |
|---|---|---|---|---|---|
| Example | Adsorbent | (%) | (° C.) | (h) | (%) |
| 5 | Amberlite | 3 | 90 | 4 | 4 |
| | IRA 401 | | | | |
| 6 | Amberlite | 5 | 90 | 4 | 9 |
| | IRA 402 | | | | |
| 7 | Amberlite | 3 | 90 | 4 | 12 |
| | IRA 900 | | | | |
| 8 | Funct. | 1 | 90 | 4 | 3 |
| | Silica gel | | | | |

1. A process for the separation of palladium from a crude reaction mixture comprising aryl acetic acids of the general formula 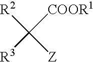
wherein
Z means phenyl, napht-2-yl, 9H-fluoren-2-yl, substituted carbazol-2-yl, benzoxazol-5-yl, either of which can be substituted with H, C₁-C₈-alkyl or C₁-C₈-cycloalkyls optionally substituted with —F or —Cl, C₆-C₁₀-aryl optionally substituted with F or Cl, OR⁴, COR⁵, —F, —Cl; and pyrrolyl or dehydropyrrolyl or 1-oxo-1,3-dehydro-isoindol-2-yl optionally substituted with F or Cl, OR⁴, COR⁵, —F, —Cl;
R1 means H or C₁-C₄-alkyl; and R², R³, R⁴, R⁵ mean independently of each other; H, C₁-C₈ alkyl, C₆-C₁₀-aryl optionally substituted with —F or —Cl, or thiophenyl;
wherein the crude reaction mixture is obtained by palladium catalyzed carbonylation by adsorption of the palladium on a solid adsorbent, characterized in that the adsorption is carried out in the absence of a reducing agent for palladium and at a temperature, in which the crude reaction mixture is molten.

2. A process as claimed in claim 1, wherein said crude reaction mixture comprises a compound selected from the group consisting of ibuprofen, naproxen, ketoprofen, flurbiprofen, indoprofen, suprofen, hexaprofen, pirprofen, fenoprofen, cicloprofen, mexoprofen, benoxaprofen and carprofen.

3. A process as claimed in claim 1, wherein said crude reaction mixture comprises ibuprofen or naproxen.

4. A process as claimed in claim 1, wherein the solid adsorbent is selected from the group consisting of activated carbon, optionally functionalized silica gel, aluminum oxide, infusorial earth, magnesium oxide, ion-exchange resin, neutral solid adsorbent, zeolite and a combination of two or more of such solid adsorbents.

5. A process as claimed in claim 1, wherein the adsorbent is directly added to the crude reaction mixture, stirred and then separated by filtration.

6. A process as claimed in claim 1, wherein the adsorption is conducted at a temperature in the range of 40° C. to 180° C.

7. A process as claimed in claim 1, wherein the said temperature is in the range of 40° C. to 150° C.

8. A process as claimed in claim 1, wherein the temperature is in the range of 60° C. to 120° C.

9. A process as claimed in claim 1, wherein the solid adsorbent comprises activated carbon with an average particle-size of less than 150 μm for 80% of the particles.

10. A process as claimed in claim 9, wherein the activated carbon has an average particle-size of less than 60 μm for 80% of the particles.

11. A process as claimed in claim 1, wherein the solid absorbent is a basic ion-exchange resin

12. A process as claimed in claim 11, wherein the ion-exchange resin is a strong basic ion-exchange resin.

13. A process as claimed in claim 1, wherein the solid adsorbent is a functionalized silica gel.

14. A process as claimed in claim 13, wherein the said functionalized silica gel is functionalized with phosphine groups.

15. A process as claimed in claim 14, wherein the said functionalized silica gel is preferably a silica gel functionalized with diphenyl phosphine groups.

16. A process as claimed in claim 9, wherein the separation of palladium from the crude reaction mixture accounts for 85% to 96% of the palladium originally present in the crude reaction mixture.

17. A process for the separation of palladium from a crude reaction mixture containing ibuprofen, wherein the crude reaction mixture is obtained by palladium catalyzed carbonylation by adsorption of the palladium on a solid adsorbent, characterized in that the adsorption is carried out in the absence of a reducing agent for palladium and at a temperature of 40° C. to 200° C.

18. A process as claimed in claim 17, wherein the solid adsorbent comprises activated carbon with an average particle-size of less than 150 μm for 80% of the particles.

19. A process as claimed in claim 18, wherein the separation of palladium from the crude reaction mixture accounts for 85% to 96% of the palladium originally present in the crude reaction mixture.

20. A process as claimed in claim 17, wherein the solid adsorbent comprises ion exchange resins comprising quaternary ammonium groups, and the separation of palladium from the crude reaction mixture accounts for 88% to 96% of the palladium originally present in the crude reaction mixture.
